(19) United States
(12) Patent Application Publication
(10) Pub. No.: US 2008/0207524 A1
(43) Pub. Date: Aug. 28, 2008

(54) MEDICINAL PREPARATIONS FOR TREATING SEX HORMONE-DEPENDENT DISEASES

(75) Inventors: Yasutaka Igari, Hyogo (JP); Shigeru Kamei, Hyogo (JP)

Correspondence Address: FOLEY AND LARDNER LLP;SUITE 500, 3000 K STREET NW, WASHINGTON, DC, 20007, US

(73) Assignee: TAKEDA PHARMACEUTICAL COMPANY LIMITED

(21) Appl. No.: 11/941,356

(22) Filed: Nov. 16, 2007

Related U.S. Application Data
(63) Continuation of application No. 10/312,998, filed on Jan. 2, 2003, now abandoned.

(30) Foreign Application Priority Data
Jul. 5, 2000 (JP) 2000-208253

(51) Int. Cl. A61K 38/08; A61K 38/02; A61K 31/519; A61K 31/4365; A61P 5/24

(52) U.S. Cl. 514/15; 514/2; 514/260.1; 514/301

(57) ABSTRACT

Medicinal preparations for treating sex hormone-dependent diseases which comprise a combination of a compound having a luteinizing hormone-releasing hormone agonistic effect or its salt with a compound having a luteinizing hormone-releasing hormone antagonistic effect or its salt for administering the compound having a luteinizing hormone-releasing hormone agonistic effect or its salt followed by the compound having a luteinizing hormone-releasing hormone antagonistic effect or its salt. By using these preparations, the concentration of a sex hormone (for example, testosterone, LH, FSH, estrogen) can be quickly recovered after the medicable period of a compound having a luteinizing hormone-releasing hormone antagonistic effect or its salt or a preparation containing the same (preferably a sustained-release preparation), which makes it possible to definitely determine the drug cessation period in an intermittent treatment.

### CROSS-REFERENCE TO RELATED APPLICATIONS

This application is a Continuation of U.S. application Ser. No. 10/312,998, which is the US National Stage application of PCT/JP01/05808, filed Jul. 4, 2001, which claims priority from Japanese application JP 2000-208253, filed May 7, 2000.

### TECHNICAL FIELD

The present invention relates to (1) a medicinal preparation for treating sex hormone-dependent diseases comprising a combination of a compound having a luteinizing hormone-releasing hormone agonistic effect or a salt thereof with a compound having a luteinizing hormone-releasing hormone antagonistic effect or a salt thereof, for administering the compound having a luteinizing hormone-releasing hormone agonistic effect or a salt thereof followed by the compound having a luteinizing hormone-releasing hormone antagonistic effect or a salt thereof, (2) a method for using a compound having a luteinizing hormone-releasing hormone agonistic effect or a salt thereof and/or a compound having a luteinizing hormone-releasing hormone antagonistic effect or a salt thereof for the preparation of a pharmaceutical composition for treating sex hormone-dependent diseases, said composition being characterized by administering the compound having a luteinizing hormone-releasing hormone agonistic effect or a salt thereof followed by the compound having a luteinizing hormone-releasing hormone antagonistic effect or a salt thereof, and the like.

### BACKGROUND ART

A luteinizing hormone-releasing hormone which is known as LHRH (or GnRH) is released from hypothalamus and binds to a receptor of glandula pituitaria. LH (luteinizing hormone) and FSH (follicle stimulating hormone) which are released due to this act on gonadal gland to synthesize a steroid sex hormone. The successive administration of a compound having a potent luteinizing hormone-releasing hormone agonistic effect results in the reduction of the number of the available receptor and the formation of a steroid sex hormone derived from gonadal gland is suppressed. By utilizing this, a compound having a luteinizing hormone-releasing hormone agonistic effect is clinically applied as medicaments for treatment of sex hormone-dependent diseases such as prostate cancer, benign prostatic hyperplasia, endometriosis, hysteromyoma, metrofibroma, precocious puberty, breast cancer, and the like.

As a treatment method comprising preventing and delaying the change of sex hormone-dependent diseases (especially prostate cancer) into sex hormone-independent, an intermittent treatment comprising administration cessation of a compound having a luteinizing hormone-releasing hormone agonistic effect for a drug starving, and exposing to a sex hormone with a sufficient concentration so as to maintain the disease being hormone-dependent in a treatable state, is used (for example, Cancer, 71 (1993) 2782-2790).

As a compound having a luteinizing hormone-releasing hormone agonistic effect is an agonist for LHRH receptor, the concentrations of testosterone and estrogen are increased immediately after the first administration of a preparation of the compound, due to the glandula pituitaria gonad-stimulating effect which is inherent in the compound, and a temporary deterioration of a disease (flare phenomenon) is observed. After that, the number of the LHRH receptor is decreased and the treatment of the hormone dependent diseases becomes to be effective, while the number of LHRH receptor is not recovered quickly as long as the concentration of the compound in blood is more than a certain concentration, and therefore an excessive period is required in order to be exposed to a hormone having the concentration required for an intermittent treatment. Then, it has been desired to develop a treatment method which can definitely determine the drug cessation period in an intermittent treatment; or a treatment method in which the suppression of a sex hormone is achieved during the drug administration period, while more definite recovery of the sex hormone is accelerated after finishing the administration period.

### DISCLOSURE OF THE INVENTION

The present inventors have studied intensively to solve the above-mentioned problem. As a result, the present invention has been completed.

That is, the present invention relates to:

(1) A medicinal preparation for treating sex hormone-dependent diseases comprising a combination of a compound having a luteinizing hormone-releasing hormone agonistic effect or a salt thereof with a compound having a luteinizing hormone-releasing hormone antagonistic effect or a salt thereof, for administering the compound having a luteinizing hormone-releasing hormone agonistic effect or a salt thereof followed by the compound having a luteinizing hormone-releasing hormone antagonistic effect or a salt thereof;

(2) The preparation according to the above (1), wherein the compound having a luteinizing hormone-releasing hormone agonistic effect or a salt thereof is a peptide compound or a salt thereof;

(3) The preparation according to the above (2), wherein the peptide compound is a natural hormone or an analog thereof;

(4) The preparation according to the above (2), wherein the peptide compound is a peptide represented by the formula:

5-oxo-Pro-His-Trp-Ser-Tyr-Y-Leu-Arg-Pro-Z

wherein Y is a residue selected from DLeu, DAla, DTrp, DSer(tBu), D2NaI and DHis(ImBzl), Z is NH—C₂H₅ or Gly-NH₂;

(5) The preparation according to the above (2), wherein the peptide compound or a salt thereof is a compound or a salt thereof selected from leuprorelin, gonadorelin, buserelin, triptorelin, goserelin, nafarelin, histrelin, deslorelin, meterelin and recirelin;

(6) The preparation according to the above (1), wherein the compound having a luteinizing hormone-releasing hormone agonistic effect or a salt thereof is used as an injectable preparation;

(7) The preparation according to the above (1), wherein the compound having a luteinizing hormone-releasing hormone agonistic effect or a salt thereof is used as a sustained release preparation;

(8) The preparation according to the above (1), wherein the compound having a luteinizing hormone-releasing hormone agonistic effect or a salt thereof is used as a preparation for nasal administration;

(9) The preparation according to the above (1), wherein the compound having a luteinizing hormone-releasing hormone antagonistic effect or a salt thereof is a peptide compound or a salt thereof;

(10) The preparation according to the above (9), wherein the peptide compound having a luteinizing hormone-releasing hormone antagonistic effect or a salt thereof is a compound selected from NAcD2NaI-D4ClPhe-D3 Pal-Ser-NMeTyr-DLys(Nic)-Leu-Lys(Nisp)-Pro-DAlaNH₂, N(4H2-furoyl)Gly-D2NaI-D4ClPhe-D3 Pal-Ser-NMeTyr-DLys(Nic)-Leu-Lys(Nisp)-Pro-DAlaNH₂, cetrorelix, ganirelix, antarelix, decirelix, azaline, antide, ramorelix and abarelix, or a salt thereof;

(11) The preparation according to the above (1), wherein the compound having a luteinizing hormone-releasing hormone antagonistic effect or a salt thereof is used as an injectable preparation;

(12) The preparation according to the above (1), wherein the compound having a luteinizing hormone-releasing hormone antagonistic effect or a salt thereof is used as a sustained release preparation;

(13) The preparation according to the above (1), wherein the compound having a luteinizing hormone-releasing hormone antagonistic effect or a salt thereof is used as a preparation for nasal administration;

(14) The preparation according to the above (1), wherein the compound having a luteinizing hormone-releasing hormone antagonistic effect or a salt thereof is a nonpeptide compound or a salt thereof;

(15) The preparation according to the above (14), wherein the nonpeptide compound or a salt thereof is a compound represented by the formula (I):

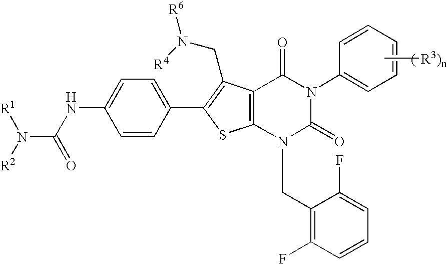

wherein each of R¹ and R² is hydrogen atom, hydroxy group, a C₁₋₄ alkoxy group, a C₁₋₄ alkoxy-carbonyl group or an optionally substituted C₁₋₄ alkyl group,
R³ is hydrogen atom, a halogen atom, hydroxy group or an optionally substituted C₁₋₄ alkoxy group, or the two adjacent R³s may link to form a C₁₋₄ alkylenedioxy group,
R⁴ is hydrogen atom or a C₁₋₄ alkyl group,
R⁶ is an optionally substituted C₁₋₄ alkyl group or a group represented by the formula:

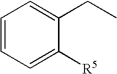

wherein R⁵ is hydrogen atom, or R⁴ and R⁵ may link to form a heterocyclic ring, and
n is an integer of 0 to 5, or a salt thereof;

(16) The preparation according to the above (14), wherein the nonpeptide compound or a salt thereof is 5-(N-benzyl-N-methylaminomethyl)-1-(2,6-difluorobenzyl)-6-[4-(3-methoxyureido)phenyl]-3-phenylthieno[2,3-d]pyrimidine-2,4-(1H,3H)-dione or a salt thereof;

(17) The preparation according to the above (14), wherein the nonpeptide compound or a salt thereof is a compound represented by the formula (VIII):

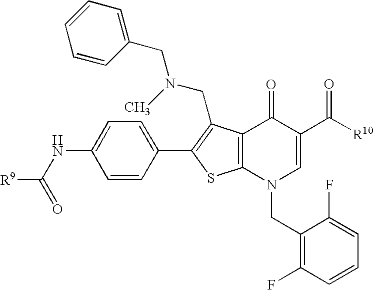

wherein R⁹ is an optionally substituted C₁₋₇ alkyl group, an optionally substituted C₃₋₇ cycloalkyl group, an optionally substituted C₁₋₆ alkoxyamino group or an optionally substituted hydroxyamino group,
R¹⁰ is an optionally substituted C₁₋₇ alkyl group or an optionally substituted phenyl group, or a salt thereof;

(18) The preparation according to the above (14), wherein the nonpeptide compound or a salt thereof is 3-(N-benzyl-N-methylaminomethyl)-4,7-dihydro-5-isobutylyl-7-(2,6-difluorobenzyl)-2-[4-[(1-hydroxycyclopropyl)-carbonylamino]phenyl]-4-oxothieno[2,3-b]pyridine or a salt thereof;

(19) The preparation according to the above (1), wherein the compound having a luteinizing hormone-releasing hormone antagonistic effect or a salt thereof is administrated before administration, during administration or immediately after administration of the compound having a luteinizing hormone-releasing hormone agonistic effect or a salt thereof;

(20) A method for using a compound having a luteinizing hormone-releasing hormone agonistic effect or a salt thereof and/or a compound having a luteinizing hormone-releasing hormone antagonistic effect or a salt thereof for the preparation of a medicinal preparation for treating sex hormone-dependent diseases, said preparation being characterized by administering the compound having a luteinizing hormone-releasing hormone agonistic effect or a salt thereof, followed by the compound having a luteinizing hormone-releasing hormone antagonistic effect or a salt thereof;

(21) A medicinal preparation for treating sex hormone-dependent diseases comprising a compound having a luteinizing hormone-releasing hormone agonistic effect or a salt thereof or a compound having a luteinizing hormone-releasing hormone antagonistic effect or a salt thereof, said preparation being characterized by administering the compound having a luteinizing hormone-releasing hormone agonistic effect or a salt thereof, followed by the compound having a luteinizing hormone-releasing hormone antagonistic effect or a salt thereof;

(22) An agent for maintaining a hormone therapy comprising a compound having a luteinizing hormone-releasing hormone antagonistic effect or a salt thereof;

(23) A method for determining a drug cessation period comprising using a compound having a luteinizing hormone-releasing hormone antagonistic effect or a salt thereof;

(24) A method for recovering the concentration of a sex hormone in a living body comprising using a compound having a luteinizing hormone-releasing hormone antagonistic effect or a salt thereof;

(25) A method for the treatment of sex hormone-dependent diseases comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal;

(26) A method for maintaining hormone therapy comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal;

(27) A method for determining a drug cessation period comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal;

(28) A method for recovering sex hormone concentration in a living body, comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal;

(29) A method for treating a sex hormone-dependent disease, comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal, thereby maintaining sex hormone-dependency; and the like.

### BEST MODE FOR CARRYING OUT THE INVENTION

In the present description, the sex hormone-dependent diseases mean diseases such as sex hormone-dependent cancer (e.g., prostate cancer, uterine cancer, breast cancer, glandula pituitaria tumor and the like), benign prostatic hyperplasia, endometriosis, hysteromyoma, precocious puberty, dysmenorrhea, amenorrhea, premenstrual syndrome, multilocular ovarian syndrome and the like.

The compound having luteinizing hormone-releasing hormone agonistic effect or the compound having luteinizing hormone-releasing hormone antagonistic effect used in the present invention is not specifically limited as far as it is pharmacologically useful, and may be a nonpeptide compound or a peptide compound. Preferred examples of the peptide compound include a physiologically active peptide having molecular weight of about 300 to about 40,000, preferably about 400 to about 30,000, more preferably about 500 to about 20,000, and the like.

Specifically, as the peptide compound, there are, for example, the peptides described in the Treatment with GnRH analogs: Controversies and perspectives (The Parthenon Publishing Group Ltd., published in 1996), JP 3-503165 A, JP 3-101695 A, JP 7-97334 A, JP 8-259460 A, and the like.

Specific examples of the peptide compound having a luteinizing hormone-releasing hormone agonistic effect include a peptide represented by the formula:

5-oxo-Pro-His-Trp-Ser-Tyr-Y-Leu-Arg-Pro-Z

wherein Y is a residue selected from DLeu, DAla, DTrp, DSer(tBu), D2NaI and DHis(ImBzl), Z is NH—C₂H₅ or Gly-NH₂. Especially, the peptide wherein Y is DLeu and Z is NH—C₂H₅ (i.e., the peptide represented by 5-oxo-Pro-His-Trp-Ser-Tyr-DLeu-Leu-Arg-Pro-NH—C₂H₅; leuprorelin) or an acetate thereof is preferred.

Furthermore, examples of the peptide compound having a luteinizing hormone-releasing hormone agonistic effect specifically include gonadorelin, buserelin, triptorelin, goserelin, nafarelin, histrelin, deslorelin, meterelin, recirelin and the like.

Specific examples of the peptide compound having a luteinizing hormone-releasing hormone antagonistic effect specifically include a peptide represented by the formula:

X-D2NaI-D4ClPhe-D3 Pal-Ser-A-B-Leu-C-Pro-DAlaNH₂

wherein X is N(4H₂-furoyl)Gly or NAc, A is a residue selected from NMeTyr, Tyr, Aph(Atz), NMeAph(Atz), B is a residue selected from DLys(Nic), DCit, DLys(AzaglyNic), DLys(AzaglyFur), DhArg(Et₂), DAph(Atz) and DhCi, and C is Lys(Nisp), Arg or hArg(Et₂), and the like. Specifically, the peptide wherein X is NAc, A is NMeTyr, B is DLys(Nic) and C is Lys(Nisp) (i.e., the peptide represented by NAcD2NaI-D4ClPhe-D3 Pal-Ser-NMeTyr-DLys(Nic)-Leu-Lys(Nisp)-Pro-DAlaNH₂) is preferred.

Furthermore, specific examples of the peptide compound having a luteinizing hormone-releasing hormone antagonistic effect include, NAcD2NaI-D4ClPhe-D3 Pal-Ser-NMeTyr-DLys(Nic)-Leu-Lys(Nisp)-Pro-DAlaNH₂, N(4H2-furoyl)Gly-D2NaI-D4ClPhe-D3 Pal-Ser-NMeTyr-DLys(Nic)-Leu-Lys(Nisp)-Pro-DAlaNH₂, cetrorelix, ganirelix, antarelix, decirelix, azaline, antide, ramorelix, abarelix, and the like.

These peptides can be prepared by methods as described the above-mentioned literatures or patent publications or similar methods thereof.

The compound having a luteinizing hormone-releasing hormone agonistic effect or the compound having a luteinizing hormone-releasing hormone antagonistic effect used in the present invention may be the free compound itself or a pharmacologically acceptable salt thereof.

Examples of the salt include, when the compound having a luteinizing hormone-releasing hormone agonistic effect or the compound having a luteinizing hormone-releasing hormone antagonistic effect has a basic group such as an amino group and the like, a salt with an inorganic acid (also referred to an inorganic free acid) (e.g., carbonic acid, bicarbonic acid, hydrochloric acid, sulfuric acid, nitric acid, boric acid and the like), an organic acid (also referred to an organic free acid) (e.g., succinic acid, acetic acid, propionic acid, trifluoroacetic acid and the like), and the like.

When the biologically active substance has an acidic group such as a carboxyl group and the like, examples of the salt includes a salt with an inorganic base (also referred to an inorganic free base) (e.g., an alkaline metal such as sodium, potassium and the like, an alkaline earth metal such as calcium, magnesium and the like, and the like) or an organic base (also referred to an organic free base) (e.g., an organic amine such as triethylamine and the like, a basic amino acid such as arginine and the like). Further, the physiologically active peptide may form a metal complex compound (e.g., copper complex, zinc complex and the like).

Specifically, both of the above-mentioned peptide represented by NAcD2NaI-D4ClPhe-D3 Pal-Ser-NMeTyr-DLys(Nic)-Leu-Lys(Nisp)-Pro-DAlaNH₂ and the peptide represented by 5-oxo-Pro-His-Trp-Ser-Tyr-DLeu-Leu-Arg-Pro-NH—C₂H₅ (leuprorelin) are preferably used as acetates.

Furthermore, as a nonpeptide compound having a luteinizing hormone-releasing hormone antagonistic effect, any of compounds having a luteinizing hormone-releasing hormone antagonistic effect may be used. For example, in addition to the above-mentioned compound (I), the compound (VIII) and the like, there are the compounds described in WO99/21553, WO99/21557, WO99/41251, WO99/41252, WO99/51231, WO99/51232, WO99/51233, WO99/51234, WO99/51595, WO99/51596, WO99/44987, WO99/50275, WO99/50276, WO00/04013, WO00/12521, WO00/12522, WO00/29380, WO00/20358 and the like.

Hereinafter, the definition of the substituents in the above-mentioned formula (I) is described.

Examples of the “C₁₋₄ alkoxy group” represented by R¹ or R² include methoxy, ethoxy, propoxy, isopropoxy, butoxy, tert-butoxy and the like. Among these, a C₁₋₃ alkoxy group is preferred, and methoxy is more preferred.

Examples of the “C₁₋₄ alkoxy-carbonyl group” represented by R¹ or R² include methoxycarbonyl, ethoxycarbonyl, propoxycarbonyl, isopropoxycarbonyl, butoxycarbonyl, tert-butoxycarbonyl and the like. Among these, a C₁₋₃ alkoxy-carbonyl group is preferred, and methoxycarbonyl is more preferred.

Examples of the “C₁₋₄ alkyl group” of the “optionally substituted C₁₋₄ alkyl group” represented by R¹ or R² include a linear C₁₋₄ alkyl group (e.g., methyl, ethyl, propyl, butyl and the like), a branched C₃₋₄ alkyl group (e.g., isopropyl, isobutyl, sec-butyl, tert-butyl and the like) and the like. Among these, a C₁₋₃ alkyl group is preferred. Especially, ethyl is preferred.

Examples of the “substituent” of the “optionally substituted C₁₋₄ alkyl group” represented by R¹ or R² include (i) hydroxy, (ii) a C₁₋₇ acyloxy (e.g., a C₁₋₆ alkyl-carbonyloxy such as acetoxy, propionyloxy and the like), (iii) benzoyloxy, (iv) an amino group optionally having 1 or 2 substituent(s) selected from a C₁₋₆ alkoxy-carbonyl (e.g., methoxycarbonyl, ethoxycarbonyl, tert-butoxycarbonyl and the like), benzyloxycarbonyl, a C₁₋₄ acyl (e.g., a C₁₋₃ alkyl-carbonyl such as acetyl, propionyl and the like), a C₁₋₄ alkyl (e.g., methyl, ethyl, propyl, butyl and the like) and a C₁₋₃ alkylsulfonyl (e.g., methanesulfonyl and the like) and the like (e.g., amino, dimethylamino, methoxycarbonylamino, ethoxycarbonylamino, tert-butoxycarbonylamino, benzyloxycarbonylamino, acetylamino, methanesulfonylamino and the like), (v) a C₁₋₁₀ alkoxy (e.g., methoxy, ethoxy, propoxy, tert-butoxy and the like), (vi) a C₃₋₇ cycloalkyloxycarbonyl-C₁₋₃ alkoxy (e.g., cyclohexyloxycarbonyloxy-1-ethoxy and the like), (vii) a C₁₋₃ alkoxy-C₁₋₃ alkoxy (e.g., methoxymethoxy, methoxyethoxy and the like) and the like. Among these, hydroxy is preferred.

The “C₁₋₄ alkyl group” of the “optionally substituted C₁₋₄ alkyl group” represented by R¹ or R² may have, for example, 1 to 5, preferably 1 to 3 of the above-mentioned substituent(s) at the substitutable position(s), and when the number of the substituents is two or more, the substituents may be the same or different.

Preferably, one of R¹ and R² is hydrogen atom and the other is a C₁₋₃ alkoxy group.

Examples of the “halogen atom” represented by R³ include fluorine, chlorine, bromine, iodine. Among these, chlorine is preferred.

Examples of the “C₁₋₄ alkoxy group” of the “optionally substituted C₁₋₄ alkoxy group” represented by R³ include methoxy, ethoxy, propoxy, isopropoxy, butoxy, tert-butoxy and the like. Among these, methoxy is preferred.

Examples of the “substituent” of the “optionally substituted C₁₋₄ alkoxy group” represented by R³ include the substituents same as the “substituent” of the “optionally substituted C₁₋₄ alkyl group” represented by the above-mentioned R¹ or R². Among these, a C₁₋₄ alkoxy group is preferred.

The C₁₋₄ alkoxy group may have, for example, 1 to 5, preferably 1 to 3 of the above-mentioned substituent(s) at the substitutable position(s), and when the number of the substituents is two or more, the substituents may be the same or different.

Examples of the “C₁₋₄ alkylenedioxy group” formed by the two adjacent R³s include methylenedioxy, ethylenedioxy and the like.

R³ is preferably hydrogen atom.

Examples of the “C₁₋₄ alkyl group” represented by R⁴ include a linear C₁₋₄ alkyl group (e.g., methyl, ethyl, propyl, butyl and the like), a branched C₃₋₄ alkyl group (e.g., isopropyl, isobutyl, sec-butyl, tert-butyl and the like) and the like. Among these, a C₁₋₃ alkyl group is preferred. Especially, methyl is preferred.

Examples of the “optionally substituted C₁₋₄ alkyl group” represented by R⁶ include the “optionally substituted C₁₋₄ alkyl group” represented by R¹ or R².

Examples of the “heterocyclic ring” which is formed by the linking of R⁴ and R⁵ include a 5- or 6-membered nitrogen-containing heterocyclic group. When R⁴ and R⁵ are linked, a group represented by the formula:

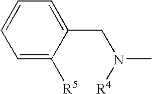

include, for example, a group represented by the formula:

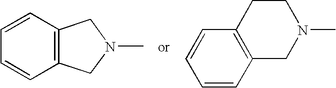

and the like. Among these, a group represented by the formula:

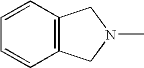

is preferred.

R⁶ is preferably a group represented by the formula:

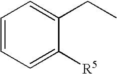

wherein R⁵ is as defined above.

Preferably, R⁴ is a C₁₋₃ alkyl group and R⁵ is hydrogen atom.

“n” is preferably an integer of 0 to 2.

Preferred examples of the compound (I) include the compound wherein R¹ is hydroxy group, methoxy group or a C₁₋₃ alkyl group; R² is hydrogen atom or a C₁₋₃ alkyl group; R⁴ is a C₁₋₃ alkyl group; R⁶ is benzyl group; and n is 0 or a salt thereof.

Among these, the compound wherein R¹ is an C₁₋₃ alkoxy group; each of R² and R⁵ is hydrogen atom; R⁴ is a C₁₋₃ alkyl group; R⁶ is a benzyl group; and n is 0 or a salt thereof is preferred.

The specific examples of the compound (I) include,
5-(N-benzyl-N-methylaminomethyl)-1-(2,6-difluorobenzyl)-6-[4-(3-methoxyureido)phenyl]-3-phenylthieno[2,3-d]pyrimidine-2,4-(1H,3H)-dione,
5-(N-benzyl-N-methylaminomethyl)-1-(2,6-difluorobenzyl)-6-[4-(3-hydroxyureido)phenyl]-3-phenylthieno[2,3-d]pyrimidine-2,4-(1H,3H)-dione,
5-(N-benzyl-N-methylaminomethyl)-1-(2,6-difluorobenzyl)-6-[4-(3-methylureido)phenyl]-3-phenylthieno[2,3-d]pyrimidine-2,4-(1H,3H)-dione,
5-(N-benzyl-N-methylaminomethyl)-1-(2,6-difluorobenzyl)-6-[4-(3-ethylureido)phenyl]-3-phenylthieno[2,3-d]pyrimidine-2,4-(1H,3H)-dione,
   or a salt thereof.

Among these, 5-(N-benzyl-N-methylaminomethyl)-1-(2,6-difluorobenzyl)-6-[4-(3-methoxyureido)phenyl]-3-phenylthieno[2,3-d]pyrimidine-2,4-(1H,3H)-dione or a salt thereof is preferred.

Hereinafter, the definition of the substituents in the above-mentioned formula (VIII) is described.

Examples of the “C₁₋₇ alkyl group” of the “optionally substituted C₁₋₇ alkyl group” represented by R⁹ include a linear C₁₋₇ alkyl group (e.g., methyl, ethyl, propyl, butyl, pentyl, hexyl, heptyl and the like), a branched C₃₋₇ alkyl group (e.g., isopropyl, isobutyl, sec-butyl, tert-butyl, isopentyl, neopentyl and the like) and the like. Among these, a branched C₃₋₇ alkyl group is preferred. Especially, isopropyl is preferred.

Examples of the “substituent” of the “optionally substituted C₁₋₇ alkyl group” represented by R⁹ include (i) a hydroxy group, (ii) a C₁₋₇ acyloxy (e.g., a C₁₋₆ alkyl-carbonyloxy such as acetoxy, propionyloxy and the like; benzoyloxy and the like), (iii) an amino optionally having 1 or 2 substituent(s) selected from a C₁₋₆ alkoxy-carbonyl (e.g., methoxycarbonyl, ethoxycarbonyl, tert-butoxycarbonyl and the like), benzyloxycarbonyl, a C₁₋₃ acyl (e.g., a C₁₋₂ alkyl-carbonyl such as acetyl, propionyl and the like, and the like), a C₁₋₃ alkylsulfonyl (e.g., methanesulfonyl and the like) and a C₁₋₃ alkyl (e.g., methyl, ethyl and the like) (specific examples: amino, methoxycarbonylamino, ethoxycarbonylamino, tert-butoxycarbonylbenzyloxycarbonylamino, acetylamino, methanesulfonylamino, methylamino, dimethylamino and the like), (iv) a C₁₋₁₀ (preferably C₁₋₄) alkoxy optionally having 1 to 3 substituent(s) selected from a C₃₋₇ cycloalkyloxycarbonyl (e.g., cyclohexyloxycarbonyloxy and the like) and a C₁₋₃ alkoxy (e.g., methoxy, ethoxy and the like) (e.g., methoxy, ethoxy, propoxy, tert-butoxy, cyclohexyloxycarbonyloxy-1-ethoxy, methoxymethoxy, ethoxymethoxy and the like), (v) a C₁₋₆ alkoxy-carbonyl (e.g., methoxycarbonyl, ethoxycarbonyl, propoxycarbonyl and the like) and the like. Among these, hydroxy group is preferred.

The “C₁₋₇ alkyl group” may have, for example, 1 to 5, preferably 1 to 3 of the above-mentioned substituent(s) at the substitutable position(s), and when the number of the substituents is two or more, the substituents may be the same or different.

Examples of the “C₃₋₇ cycloalkyl group” of the “optionally substituted C₃₋₇ cycloalkyl group” represented by R⁹ include cyclopropyl, cyclobutyl, cyclopentyl, cyclohexyl, cycloheptyl and the like. Among these, cyclopropyl is preferable.

Examples of the “substituent” of the “optionally substituted C₃₋₇ cycloalkyl group” represented by R⁹ include the same 1 to 3 substituents as the above-mentioned “substituent” of the “optionally substituted C₁₋₇ alkyl group” represented by R⁹. When the number of the substituents is two or more, the substituents may be the same or different.

Examples of the “C₁₋₆ alkoxyamino group” of the “optionally substituted C₁₋₆ alkoxyamino group” represented by R⁹ include a mono- or di-C₁₋₆ alkoxyamino group (e.g., methoxyamino, ethoxyamino, dimethoxyamino, diethoxyamino, ethoxymethoxyamino and the like). Among these, a mono-C₁₋₃ alkoxyamino group (e.g., methoxyamino and the like) is preferred.

Examples of the “substituent” of the “optionally substituted C₁₋₆ alkoxyamino group” represented by R⁹ include the same number and kind of the substituents as the above-mentioned “substituent” of the “optionally substituted C₁₋₇ alkyl group” represented by R⁹. When the number of the substituents is two or more, the substituents may be the same or different. The “substituent” may replace the “C₁₋₆ alkoxy group” of the C₁₋₆ alkoxyamino group or the “nitrogen atom of the amino group”.

Specific examples of the “optionally substituted C₁₋₆ alkoxyamino group” include methoxyamino, N-methyl-N-methoxyamino, N-ethyl-N-methoxyamino, ethoxyamino, dimethoxyamino, diethoxyamino, ethoxymethoxyamino and the like. The preferred examples include, a C₁₋₃ alkoxyamino group, N—C₁₋₃ alkyl-N—C₁₋₃ alkoxyamino group and the like.

The “substituent” of the “optionally substituted hydroxyamino group” represented by R⁹ may replace the “hydroxy group” of the hydroxyamino group or the “nitrogen atom of the amino group”, and examples of the substituent on the “hydroxy group” include (i) a C₁₋₇ acyloxy group (e.g., a C₁₋₆ alkyl-carbonyloxy such as acetoxy, propionyloxy and the like; benzoyloxy and the like), (ii) an amino group optionally having 1 or 2 substituent(s) selected from a C₁₋₆ alkoxy-carbonyl (e.g., methoxycarbonyl, ethoxycarbonyl, tert-butoxycarbonyl and the like), benzyloxycarbonyl, a C₁₋₃ acyl (e.g., a C₁₋₂ alkyl-carbonyl such as acetyl, propionyl and the like, and the like), a C₁₋₃ alkylsulfonyl (e.g., methanesulfonyl and the like) and a C₁₋₃ alkyl (e.g., methyl, ethyl and the like) and the like (specific examples: amino, methoxycarbonylamino, ethoxycarbonylamino, tert-butoxycarbonylbenzyloxycarbonylamino, acetylamino, methanesulfonylamino, methylamino, dimethylamino and the like), (iii) a C₁₋₁₀ (preferably C₁₋₄) alkoxy group optionally having 1 to 3 substituent(s) selected from a C₃₋₇ cycloalkyloxycarbonyl (e.g., cyclohexyloxycarbonyloxy and the like) and a C₁₋₃ alkoxy (e.g., methoxy, ethoxy and the like) (e.g., methoxy, ethoxy, propoxy, tert-butoxy, cyclohexyloxycarbonyloxy-1-ethoxy, methoxymethoxy, ethoxymethoxy and the like) and the like; and examples of the substituents on the “nitrogen atom of the amino group” include the groups described in the above-mentioned (i)-(iii) as well as a C₁₋₆ alkyl group (e.g., methyl, ethyl, propyl, isopropyl, butyl, isobutyl, sec-butyl, tert-butyl, pentyl, isopentyl, neopentyl, hexyl and the like) and the like. The number of the substituents of the hydroxyamino group is generally 1 to 5, preferably 1 to 3. When the number of the substituents is two or more, the substituents may be the same or different.

Preferred examples of the “optionally substituted hydroxyamino group” include, a N—C₁₋₆ alkyl-N-hydroxyamino group (e.g., N-methyl-N-hydroxyamino, N-ethyl-N-hydroxyamino and the like) and the like. N—C₁₋₃ alkyl-N-hydroxyamino group or the like is more preferable.

Examples of the “C₁₋₇ alkyl group” of the “optionally substituted C₁₋₇ alkyl group” represented by R¹⁰ include a linear or branched C₁₋₇ alkyl group (e.g., methyl, ethyl, propyl, isopropyl, butyl, isobutyl, sec-butyl, tert-butyl, pentyl, isopentyl, neopentyl, hexyl, heptyl and the like) and the like. Among these, a C₁₋₃ alkyl group (e.g., methyl, ethyl, propyl, isopropyl and the like) is preferred. Especially isopropyl is preferred.

Examples of the “substituent” of the “optionally substituted C₁₋₇ alkyl group” represented by R¹⁰ include the same number and kind of the substituents as the above-mentioned “substituent” of the “optionally substituted C₁₋₇ alkyl group” represented by R⁹. When the number of the substituents is two or more, the substituents may be the same or different.

Examples of the “substituent” of the “optionally substituted phenyl group” represented by R¹⁰ include a halogen (e.g., fluorine, chlorine, bromine, iodine and the like), a C₁₋₃ alkyl group (e.g., methyl, ethyl, propyl, isopropyl and the like), a C₁₋₃ alkoxy group (for example, methoxy, ethoxy, propoxy, isopropoxy and the like). Among these, a halogen (preferably fluorine) is preferred.

The “phenyl group” may have, for example, 1 to 5, preferably 1 to 3 of the above-mentioned substituent(s) at the substitutable position(s), and when the number of the substituents is two or more, the substituents may be the same or different.

R⁹ is preferably a substituted branched C₃₋₇ alkyl group or a substituted C₃₋₇ cycloalkyl group, more preferably, a C₁₋₇ alkyl group substituted with a hydroxy group or a C₃₋₇ cycloalkyl group substituted by a hydroxy group. Among these, a substituted C₃₋₇ cycloalkyl group is preferred. Also preferred are a C₁₋₃ alkyl group optionally substituted with a hydroxy group, a C₃₋₇ cycloalkyl group optionally substituted with a hydroxy group, a mono-C₁₋₃ alkoxyamino, a N—C₁₋₃ alkyl-N-hydroxyamino group, hydroxyamino group and the like. Especially preferable R⁹ is cyclopropyl group optionally substituted by hydroxy group or methoxyamino group and the like. The most preferred is cyclopropyl group substituted by hydroxy group.

R¹⁰ is preferably an optionally substituted C₁₋₇ alkyl group, and more preferably a C₁₋₃ alkyl group optionally substituted with hydroxy group. Especially, isopropyl is preferable. Also preferred is phenyl.

Preferred examples of the compound (VIII) include the compound wherein R⁹ is a C₁₋₃ alkyl group optionally substituted with hydroxy group, a C₃₋₇ cycloalkyl group optionally substituted with hydroxy group, or a mono-C₁₋₃ alkoxyamino group; R¹⁰ is a C₁₋₃ alkyl group, or a salt thereof, and the like.

More preferably, a compound wherein R⁹ is (1) a C₁₋₄ alkyl group substituted with 1 or 2 hydroxy group(s), (2) a C₃₋₇ cycloalkyl group substituted with hydroxy group, or (3) a C₁₋₃ alkoxyamino group; R¹⁰ is isopropyl group or phenyl group, or a salt thereof and the like.

The specific example of the compound (VIII) include,
3-(N-benzyl-N-methylaminomethyl)-4,7-dihydro-5-isobutylyl-7-(2,6-difluorobenzyl)-2-(4-cyclopropanecarbonyl-aminophenyl)-4-oxothieno[2,3-b]pyridine,
5-benzoyl-3-(N-benzyl-N-methylaminomethyl)-7-(2,6-difluorobenzyl)-4,7-dihydro-4-oxo-2-[4-(3-hydroxy-2-methylpropionylamino)phenyl]thieno[2,3-b]pyridine,
5-(4-fluorobenzoyl)-3-(N-benzyl-N-methylaminomethyl)-7-(2,6-difluorobenzyl)-4,7-dihydro-4-oxo-2-(4-cyclopropane-carbonylaminophenyl)thieno[2,3-b]pyridine,
3-(N-benzyl-N-methylaminomethyl)-4,7-dihydro-5-isobutylyl-7-(2,6-difluorobenzyl)-2-[4-(3-hydroxy-2-methylpropionylamino)phenyl]-4-oxothieno[2,3-b]pyridine,
3-(N-benzyl-N-methylaminomethyl)-4,7-dihydro-5-isobutylyl-7-(2,6-difluorobenzyl)-2-(4-N′-methoxyureidophenyl)-4-oxothieno[2,3-b]pyridine,
3-(N-benzyl-N-methylaminomethyl)-4,7-dihydro-5-isobutylyl-7-(2,6-difluorobenzyl)-2-[4-[(1-hydroxycyclopropyl)-carbonylamino]phenyl]-4-oxothieno[2,3-b]pyridine,
(R)-4,7-dihydro-2-[4-(3-hydroxy-2-methylpropionylamino)phenyl]-7-(2,6-difluorobenzyl)-3-(N-benzyl-N-methylaminomethyl)-5-isobutylyl-4-oxothieno[2,3-b]pyridine,
4,7-dihydro-2-[4-(2-hydroxy-2-methylpropionylamino)phenyl]-7-(2,6-difluorobenzyl)-3-(N-benzyl-N-methylaminomethyl)-5-isobutylyl-4-oxothieno[2,3-b]pyridine,
4,7-dihydro-2-[4-(3-hydroxy-3-methylbutylylamino)phenyl]-7-(2,6-difluorobenzyl)-3-(N-benzyl-N-methylaminomethyl)-5-isobutylyl-4-oxothieno[2,3-b]pyridine,
(R)-4,7-dihydro-2-[4-(2,3-hydroxypropionylamino)phenyl]-7-(2,6-difluorobenzyl)-3-(N-benzyl-N-methylaminomethyl)-5-isobutylyl-4-oxothieno[2,3-b]pyridine,
3-(N-benzyl-N-methylaminomethyl)-5-benzoyl-7-(2,6-difluorobenzyl)-4,7-dihydro-2-[4-[(1-hydroxycyclopropyl) carbonylamino]phenyl]-4-oxothieno[2,3-b]pyridine and or a salt thereof.

Among these, 3-(N-benzyl-N-methylaminomethyl)-4,7-dihydro-5-isobutylyl-7-(2,6-difluorobenzyl)-2-[4-[(1-hydroxycyclopropyl)carbonylamino]phenyl]-4-oxothieno[2,3-b]pyridine or a salt thereof is preferred.

As the salt of the compound (I) and compound (VIII), a physiologically acceptable salt is preferred. Such salt include, for example, a salt with an inorganic acid (e.g., hydrochloric acid, bromohydric acid, nitric acid, sulfuric acid, phosphoric acid and the like), or a salt with an organic acid (e.g., formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like) and the like. When the compound (I) has an acidic group, it may form a physiologically acceptable salt with an inorganic base (e.g., a salt with an alkaline metal such as sodium, potassium, calcium, magnesium or the like, or an alkaline earth metal, ammonia and the like) or an organic base (e.g., trimethylamine, triethylamine, pyridine, picoline, ethanolamine, diethanolamine, triethanolamine, dicyclohexylamine, N,N′-dibenzylethylenediamine and the like).

The compound (I) may be prepared by a method known per se, for example, the methods described in JP 9-169768 A, WO 96/24597, or a similar method thereto. Specifically, the Production Process 1 and Production Process 2 as described below are exemplified. The compounds in the reaction formulas includes a salt thereof, and the salt include the same salt as that of the compound (I), and the like.

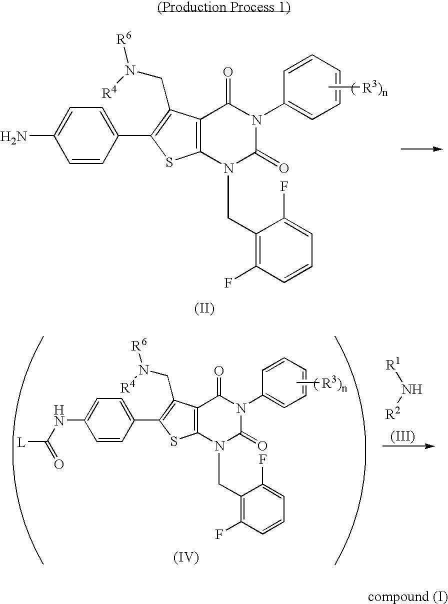

wherein L is a leaving group and each of the other symbols is as defined as above.

The “leaving group” represented by L include, for example, 1-imidazolyl, a halogen atom, an optionally substituted alkoxy group and the like. The “optionally substituted alkoxy group” include, a C₁₋₄ alkoxy group optionally having 1 to 3 halogen atom(s) (e.g., chlorine, bromine and the like) (e.g., 2,2,2-trichloroethoxy group) and the like.

The compound (II) is obtained by the method described in JP 9-169768 A or a similar method thereto.

The compound (II) and carbonyldiimidazole (N,N′-carbonyldiimidazole; CDI) or phosgene (including dimer and trimer) and the like are reacted to obtain the compound (IV), followed by reacting with the compound (III) to obtain the compound (I). The compound (IV) may be reacted without isolation or may be isolated and used in the following step.

The compound (IV) is also obtained by the reaction of the compound (II) and a chloroformic acid ester compound (e.g., 2,2,2-trichloroethyl chloroformate, 1-chloroethyl chloroformate and the like) and the like.

In the reaction of the compound (II) and carbonyldiimidazole or phosgene and the like, the amount of carbonyldiimidazole or phosgene and the like to be used is, about 1 to 3 mol per 1 mol of the compound (II).

This reaction is generally carried out in a suitable solvent which does not adversely affect the reaction.

Examples of the solvent used include ethers (e.g., ethylether, dioxane, dimethoxyethane, tetrahydrofuran and the like), aromatic hydrocarbons (e.g., benzene, toluene and the like), amides (e.g., dimethylformamide, dimethylacetoamide and the like), halogenated hydrocarbons (e.g., chloroform, dichloromethane and the like) and the like.

The reaction temperature is generally about 0 to about 150° C., preferably under room temperature (about 15 to about 25° C.). The reaction time is generally about 1 to about 36 hours.

The reaction is optionally carried out in the presence of a base.

Examples of the “base” include an inorganic base such as sodium carbonate, sodium hydrogen carbonate, potassium carbonate, potassium hydrogen carbonate, sodium hydroxide, potassium hydroxide, thallium hydroxide and the like, or an organic base such as triethylamine, pyridine and the like.

The amount of the “base” to be used is, about 2 mol to 20 mol, preferably about 5 mol to 12 mol per 1 mol of the compound (II).

The reaction conditions of the next reaction with the compound (III) may be the same as the reaction conditions of the reaction of the compound (II) and carbonyldiimidazole or phosgene. The amount of the compound (III) to be used is about 2 to 20 mol, preferably about 5 to 10 mol per 1 mol of the compound (II) or the compound (IV). The reaction temperature is generally about 0 to 150° C., preferably under room temperature (about 15 to 25° C.). The reaction time is generally about 1 to 6 hours.

The compound (III) and carbonyldiimidazole or phosgene may also reacted simultaneously with the compound (II).

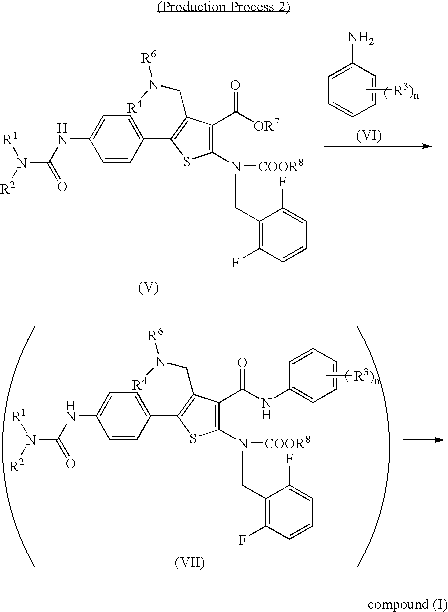

wherein R⁷ is a hydrogen atom or an alkyl group, R⁸ is an alkyl group, and each of the other symbols is as defined above.

The “alkyl group” represented by R⁷ or R⁸ include the group same as the “C₁₋₄ alkyl group” of the “optionally substituted C₁₋₄ alkyl group” represented by R¹ or R².

The compound (V) is obtained by a method known per se, for example, by reacting p-nitrophenylacetone, cyanoacetic acid ester derivertive and sulfur (e.g., Chem. Ber., Vol 99, pp. 94-100, 1966 and the like), and subjecting the obtained 2-amino-4-methyl-5-(4-nitrophenyl)thiophene to the method described in JP 9-169768 A, WO 96/24597 and the like or a similar method thereto.

(1) When R⁷ is a hydrogen atom, the compound (V) is reacted with the compound or a salt thereof (hereinafter abbreviated as compound (VI)) of the formula

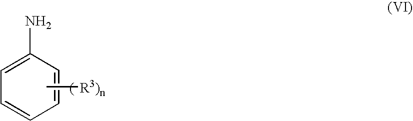

wherein each of the symbols is as defined above, in the presence of condensation reagent to obtain the compound (VII) then the obtained compound is subjected to ring-closing reaction to give the compound (I).

Examples of the “condensation reagent” include benzotriazol-1-yloxytripyrrolidinophosphonium hexafluorophosphate: PyBOP and the like.

The amount of the “condensation reagent” to be used is about 1 to 3 mol per the 1 mol of the compound (V).

The reaction is generally carried out in a suitable solvent which does not adversely affect the reaction.

As the solvent, there are, for example, alcohols (e.g., ethanol, methanol and the like), aromatic hydrocarbons (e.g., benzene, toluene and the like), amides (e.g., dimethylformamide, dimethylacetoamide and the like), halogenated hydrocarbons (e.g., chloroform, dichloromethane and the like) and the like.

The reaction temperature is generally about 0 to about 150° C., preferably under room temperature (about 15 to about 25° C.). The reaction time is generally about 1 to about 36 hours.

The product may be used in the next reaction as a reaction solution or as a crude product, or may be isolated from the reaction mixture according to a conventional method.

The compound (VII) is subjected to ring-closing reaction in the presence of a base.

As the “base”, for example, an inorganic base such as sodium methoxide, sodium carbonate, sodium hydrogen carbonate, potassium carbonate, potassium hydrogen carbonate, sodium hydroxide, potassium hydroxide, thallium hydroxide and the like, or an organic base such as triethylamine, pyridine and the like are used.

The amount of the “base” to be used is about 2 mol to 20 mol, preferably about 5 mol to 12 mol per 1 mol of the compound (VII).

The reaction is generally carried out in a suitable solvent which does not adversely affect the reaction.

The solvent used include, for example, alcohols (e.g., ethanol, methanol and the like), aromatic hydrocarbons (e.g., benzene, toluene and the like), amides (e.g., dimethylformamide, dimethylacetoamide and the like), halogenated hydrocarbons (e.g., chloroform, dichloromethane and the like) and the like.

The reaction temperature is generally about 0 to about 150° C., preferably under room temperature (about 15 to about 25° C.). The reaction time is generally about 1 to about 36 hours.

(2) When R⁷ is an alkyl group, the compound (V) is reacted with the activated compound (VI) to obtain the compound (I).

The activated compound (VI) may be prepared according to a method known per se, for example, by reacting an organic aluminum reagent and the compound (VI) in a suitable solvent which does not adversely affect the reaction.

The “organic aluminum reagent” include, for example, trimethylaluminum, dimethylaluminum chloride and the like, or a solution containing them and the like.

The amount of the “organic aluminum reagent” to be used is 1 to 5 mol, preferably 1 mol per 1 mol of the compound (VI).

As the solvent, for example, halogenated hydrocarbons (e.g., chloroform, dichloromethane and the like) is preferred.

The reaction temperature is generally about 0 to 150° C., preferably under room temperature (about 15 to 25° C.). The reaction time is generally about 1 to 6 hours.

The ring-closing reaction is carried out by the reaction of the compound (V) and the activated compound (VI) to give the compound (I).

The amount of the “compound (V)” to be used is preferably about a fifth part of the mixture of the compound (VI) and the organic aluminum reagent.

The reaction is generally carried out in a suitable solvent which does not adversely affect the reaction.

As the solvent, the solvent used in the reaction for obtaining the activated compound (VI) is preferred.

The reaction temperature is generally about 0 to 150° C., preferably under room temperature (about 15 to 25° C.). The reaction time is generally about 1 to 48 hours.

The compound (I) may be isolated and purified, by a separation means known per se, for example, recrystallization, distillation, chromatography, and the like.

When the compound (I) is obtained as a free form, it may be converted to the desired salt by a method known per se or a similar method thereto, or when obtained as a salt, it may be converted to a free form or the desired other salt by a method known per se or a similar method thereto. The compound (I) may be a hydrate or an anhydrate. The hydrate include, for example, monohydrate, 1,5-hydrate and dihydrate and the like. When the compound (I) is obtained as a mixture of optical isomers, it may be isolated as the objective(R)-form or (S)-form by a optical resolution method known per se. The compound (I) may be labelled with an isotope (e.g., ³H, ¹⁴C, ³⁵S) and the like.

The compound (VIII) or a salt thereof may be prepared by a method known per se, for example, the method described in WO95/28405, WO00/00493, or a similar method thereto.

The compound having a luteinizing hormone-releasing hormone agonistic effect or a salt thereof or the compound having a luteinizing hormone-releasing hormone antagonistic effect or a salt thereof may be used for oral administration as a tablet optionally having sugar coating, a capsule, an elixir, a sustained release preparation and the like, or an aseptic solution in water or a pharmaceutically acceptable liquid other than water, or may be used for parenteral administration as an injectable preparation such as a suspension, a sustained release preparation and the like, or as a preparation for nasal administration such as a solution, a suspension and the like. The above-mentioned preparations may be prepared, for example, by mixing the compound having a luteinizing hormone-releasing hormone agonistic effect or a salt thereof or the compound having a luteinizing hormone-releasing hormone antagonistic effect or a salt thereof with a physiologically acceptable known carrier, flavor, excipient, vehicle, antiseptic agent, stabilizer, binder or the like, in a unit dosage form according to generally required and accepted pharmaceutical practice.

The additives which may be added to a tablet, a capsule and the like include, for example, a binder such as gelatin, corn starch, tragacanth, gum arabic and the like, an excipient such as crystalline cellulose and the like, a swelling agent such as corn starch, gelatin, arginic acid and the like, a lubricant such as magnesium stearate and the like, a sweetening agent such as sucrose, lactose or saccharin and the like, a flavor such as pepper mint, Akamono oil or cherry and the like, and the like. When the preparation in the form of unit dosage is a capsule, a liquid carrier such as a fat and oil may be further added to the materials of the above-mentioned type. An aseptic injectable composition may be formulated according to a conventional pharmaceutical practice such as dissolution or suspension of an activating substance in a vehicle such as water for injection, a naturally occurring vegetable oil such as sesame oil, palm oil and the like. Examples of an aqueous injectable solution include an isotonic solution containing saline, glucose or another adjuvant (e.g., D-sorbitol, D-mannitol, sodium chloride, etc.) and the like, and may be used in combination with a suitable dissolution aid, for example, alcohol (e.g., ethanol), polyalcohol (e.g., propylene glycol, polyethylene glycol), nonionic surfactant (e.g., Polysolbate 80 (TM), HCO-50) and the like. Examples of an oily liquid include sesame oil, soybean oil and the like, and may be used in combination with benzyl benzoate, benzyl alcohol and the like, which are dissolution aids. The preparation may also be admixed with, for example, a buffer (for example, phosphorate salt buffer solution, sodium acetate buffer solution), a soothing agent (for example, benzalconium chloride, procaine hydrochloride and the like), a stabilizer (for example, human serum albumin, polyethylene glycol and the like), a preservative (for example, benzyl alcohol, phenol and the like), an antioxidant and the like. The thus-prepared injectable solution is normally filled in a suitable vial or ampoule.

Especially, the compound having a luteinizing hormone-releasing hormone agonistic effect or a salt thereof or the compound having a luteinizing hormone-releasing hormone antagonistic effect or a salt thereof is preferably used as a preparation for nasal administration, or an injectable preparation such as a sustained release preparation and the like.

The sustained release preparation (preferably sustained release injectable preparation) containing the compound having a luteinizing hormone-releasing hormone agonistic effect or a salt thereof or the compound having a luteinizing hormone-releasing hormone antagonistic effect or a salt thereof may be prepared according to a method known per se, for example, the method described in JP 60-100516 A, JP 62-201816 A, JP 4-321622 A, JP 6-192068 A, JP 9-132524 A, JP 9-221417 A, JP 11-279054 A, WO99/360099 and the like.

The oral preparation, injectable and nasal preparation of the compound having a luteinizing hormone-releasing hormone agonistic effect or a salt thereof or the compound having a luteinizing hormone-releasing hormone antagonistic effect or a salt thereof may also be prepared according to a known method or a similar method thereto.

In the present invention, the compound having a luteinizing hormone-releasing hormone agonistic effect or a salt thereof or a preparation containing the compound (preferably a sustained release preparation) is administrated, followed by administering the compound having a luteinizing hormone-releasing hormone antagonistic effect or a salt thereof or a preparation containing the compound (preferably a sustained release preparation), within the medicable period of the compound having a luteinizing hormone-releasing hormone agonistic effect or a salt thereof.

The administration interval of each compound or salts thereof or a preparation containing the compound is preferably not less than 1 day and may be suitably selected from the medicable periods of the compound having a luteinizing hormone-releasing hormone agonistic effect. For example, when a sustained release preparation containing the compound having a luteinizing hormone-releasing hormone agonistic effect or a salt thereof is used, the medicable period of the compound having a luteinizing hormone-releasing hormone agonistic effect or a salt thereof is generally not less than 7 days and up to 12 months, preferably not less than 10 days and up to 6 months, more preferably not less than 14 days and up to 4 months, and may be suitably selected from these medicable periods.

During the required treatment period, the compound having a luteinizing hormone-releasing hormone agonistic effect or a salt thereof or a preparation containing the compound (preferably a sustained release preparation) to be administrated in advance may be administered successively at administration intervals as determined by the medicable period thereof. While the compound having a luteinizing hormone-releasing hormone antagonistic effect or a salt thereof or a preparation containing the compound (preferably a sustained release preparation) is used as the final administration in a series of treatment, it may be administrated successively in order to ensure the determination of the drug cessation period.

The compound having a luteinizing hormone-releasing hormone antagonistic effect or a salt thereof or a preparation containing the compound may also be administered before administration (for example, before 1 to 3 weeks), during administration or immediately after administration (for example, after 1 to 6 hours) of the compound having a luteinizing hormone-releasing hormone agonistic effect or a salt thereof or a preparation containing the compound.

In case of the sustained release preparation, the medicable period may be determined by measurement of a sex hormone (e.g., testosterone, LH, FSH, estrogen and the like) concentration in a living body or the concentration of PSA and the like by a method known per se such as EIA, RIA and the like or a similar method thereto.

The compound having a luteinizing hormone-releasing hormone agonistic or antagonistic effect or a salt thereof may be administered to a mammal (e.g., human, monkey, dog, cow, sheep, swine, mouse, rat and the like) as a safe and low-toxic medicament.

While a dosage of the compound having a luteinizing hormone-releasing hormone agonistic or antagonistic effect or a salt thereof varies depending on an objective disease, an objective animal and the like, a dosage of a single dose may be selected from, for example, preferably the range of about 0.01 mg to 100 mg/kg body weight per an adult patient of prostate cancer. More preferably, it may be selected from the range of about 0.05 mg to 50 mg/kg body weight.

While a dosage of a sustained release preparation containing the compound having a luteinizing hormone-releasing hormone agonistic or antagonistic effect or a salt thereof varies depending on particular kind and content, particular dosage form of the compound having a luteinizing hormone-releasing hormone agonistic or antagonistic effect or a salt thereof as the basis, the release lasting time of the basis, the objective disease, the objective animal and the like, for example, in case of a sustained release preparation having a medicable period of 1 month, a dosage in a single dose of the sustained release preparation may be suitably selected from the range of, preferably about 0.1 mg to 500 mg/kg body weight per an adult patient of prostate cancer. More preferably, it may be suitably selected from the range of about 0.2 mg to 300 mg/kg body weight.

As shown in the following Example 1, when the compound having a luteinizing hormone-releasing hormone antagonistic effect or a salt thereof or a preparation containing the compound (preferably a sustained release preparation) is administered after administration of the compound having a luteinizing hormone-releasing hormone agonistic effect or a salt thereof or a preparation containing the compound (preferably a sustained release preparation), the concentration of sex hormones (e.g., testosterone, LH, FSH, estrogen and the like) is recovered quickly after the medicable period of the compound having a luteinizing hormone-releasing hormone antagonistic effect or a salt thereof. Therefore, the use method of the present invention therefore makes it possible to definitely determine the drug cessation period in an intermittent treatment.

That is, the present invention provides a method for determining a drug cessation period, and a method for recovering the concentration of a sex hormone (e.g., testosterone, LH, FSH, estrogen and the like) in a living body, wherein a preparation containing a compound having a luteinizing hormone-releasing hormone antagonistic effect or a salt thereof (preferably a sustained release preparation) is used (preferably, a combination of a preparation containing a compound having a luteinizing hormone-releasing hormone agonistic effect or a salt thereof (preferably a sustained release preparation) and a preparation containing a compound having a luteinizing hormone-releasing hormone antagonistic effect or a salt thereof (preferably a sustained release preparation) is used).

In the present invention, the drug cessation period can be determined by measuring the concentration of a sex hormone (e.g., testosterone, LH, FSH, estrogen and the like) in a living body or the concentration of PSA and the like. The drug cessation period means a period from the time when the concentration of a sex hormone in a living body is recovered to a similar concentration before administration of a preparation containing a compound having a luteinizing hormone-releasing hormone agonistic effect or a salt thereof (normal state) to the time of the next administration.

The recovery of the concentration of a sex hormone in a living body in the present invention may be determined by a criteria for measurement of a sex hormone in normal medically practice. As the concentration of a sex hormone in a living body varies depending on a particular measurement method, time for collection of blood, and the like, it is difficult to define “recover” unambiguously and numerically. However, for example, not less than about 50%, preferably not less than about 60%, more preferably not less than about 70% of the concentration before administration of a preparation containing a compound having a luteinizing hormone-releasing hormone agonistic effect or a salt thereof (normal state) is referred to be “recovered”.

Thus, by using a preparation containing a compound having a luteinizing hormone-releasing hormone antagonistic effect or a salt thereof, the concentration of a sex hormone in a living body which has been reduced by administration of a preparation containing a compound having a luteinizing hormone-releasing hormone agonistic effect or a salt thereof may be restored quickly, which makes it possible to more definitely and shortly determine the drug cessation period.

Furthermore, by using a compound having a luteinizing hormone-releasing hormone antagonistic effect or a salt thereof or a preparation containing the compound (preferably a sustained release preparation), the concentration of a sex hormone (e.g., testosterone, LH, FSH, estrogen and the like) in a living body is recovered quickly, which makes it possible to maintain the sex hormone-dependent disease being hormone-dependent due to the drug cessation period which is definitely produced. Thus, the present invention also provides an agent for maintaining a hormone therapy containing a compound having a luteinizing hormone-releasing hormone antagonistic effect or a salt thereof.

After the concentration of a sex hormone is recovered, a compound having a luteinizing hormone-releasing hormone agonistic effect or a salt thereof or a preparation containing the compound and/or a compound having a luteinizing hormone-releasing hormone antagonistic effect or a salt thereof or a preparation containing the compound may be administered.

Furthermore, the present invention relates to not only the above-mentioned:

(A) a medicinal preparation for treating sex hormone-dependent diseases comprising a combination of a compound having a luteinizing hormone-releasing hormone agonistic effect or a salt thereof with a compound having a luteinizing hormone-releasing hormone antagonistic effect or a salt thereof, for administering the compound having a luteinizing hormone-releasing hormone agonistic effect or a salt thereof followed by the compound having a luteinizing hormone-releasing hormone antagonistic effect or a salt thereof,
(B) a method for using a compound having a luteinizing hormone-releasing hormone agonistic effect or a salt thereof and/or a compound having a luteinizing hormone-releasing hormone antagonistic effect or a salt thereof for the preparation of a medicinal preparation for treating sex hormone-dependent diseases, said preparation being characterized by administering the compound having a luteinizing hormone-releasing hormone agonistic effect or a salt thereof, followed by the compound having a luteinizing hormone-releasing hormone antagonistic effect or a salt thereof,
(C) a medicinal preparation for treating sex hormone-dependent diseases comprising a compound having a luteinizing hormone-releasing hormone agonistic effect or a salt thereof or a compound having a luteinizing hormone-releasing hormone antagonistic effect or a salt thereof, wherein the compound having a luteinizing hormone-releasing hormone agonistic effect or a salt thereof is administered followed by the compound having a luteinizing hormone-releasing hormone antagonistic effect or a salt thereof, for the treatment of the sex hormone-dependent diseases,
(D) an agent for maintaining a hormone therapy comprising a compound having a luteinizing hormone-releasing hormone antagonistic effect or a salt thereof,
(E) a method for determining a drug cessation period, comprising using a compound having a luteinizing hormone-releasing hormone antagonistic effect or a salt thereof, and
(F) a method for restoration of the concentration of a sex hormone in a living body, wherein the method is carried out by using a compound having a luteinizing hormone-releasing hormone antagonistic effect or a salt thereof, but also:
(G) a pharmaceutical composition for treating sex hormone-dependent diseases comprising combination dosage forms for administration of a compound having a luteinizing hormone-releasing hormone agonistic effect or a salt thereof followed by a compound having a luteinizing hormone-releasing hormone antagonistic effect or a salt thereof, and comprising a combination of the compound having a luteinizing hormone-releasing hormone agonistic effect or a salt thereof with the compound having a luteinizing hormone-releasing hormone antagonistic effect or a salt thereof,
(H) a method for treating sex hormone-dependent diseases, comprising administering a compound having a luteinizing hormone-releasing hormone agonistic effect or a salt thereof followed by a compound having a luteinizing hormone-releasing hormone antagonistic effect or a salt thereof,
(I) a method for determining a drug cessation period in an intermittent treatment of sex hormone-dependent diseases, comprising administering a compound having a luteinizing hormone-releasing hormone agonistic effect or a salt thereof followed by a compound having a luteinizing hormone-releasing hormone antagonistic effect or a salt thereof, and determining the concentration of a hormone after the medicable period of the compound having a luteinizing hormone-releasing hormone antagonistic effect or a salt thereof,
(J) a method for treating sex hormone-dependent diseases, comprising administering a compound having a luteinizing hormone-releasing hormone agonistic effect or a salt thereof followed by a compound having a luteinizing hormone-releasing hormone antagonistic effect or a salt thereof, thereby maintaining sex hormone-dependency, and the like.

The concentration of a sex hormone (e.g., testosterone, LH, FSH, estrogen) during the medicable period or after the medicable period may be determined according to a method known per se (EIA, RIA and the like) or a similar method thereto.

Furthermore, in the use method of the present invention and the like, by administrating a compound having a luteinizing hormone-releasing hormone antagonistic effect or a salt thereof or a preparation containing the compound (preferably a sustained release preparation) before administration of a compound having a luteinizing hormone-releasing hormone agonistic effect or a salt thereof or a preparation containing the compound (preferably a sustained release preparation), the elicitation of a flare phenomenon which is observed after administration of the compound having a luteinizing hormone-releasing hormone agonistic effect or a salt thereof or the preparation containing the compound (preferably a sustained release preparation) may be prevented.

The meaning of the abbreviations as used in the present description is as follows.

### Abbreviation Name

N(4H₂-furoyl)Gly: N-tetrahydrofroylglycine residue
NAc: N-acetyl group
D2Nal: D-3-(2-naphthyl)alanine residue
D4ClPhe: D-3-(4-chloro)phenylalanine residue
D3 Pal: D-3-(3-pyridyl)alanine residue
NMeTyr: N-methyltyrosine residue
Aph(Atz): N-[5′-(3′-amino-1′H-1′,2′,4′-triazolyl)]phenylalanine residue
NMeAph(Atz): N-methyl-[5′-(3′-amino-1′H-1′,2′,4′-triazolyl)]phenylalanine residue
DLys(Nic): D-(ε-N-nicotinoyl)lysine residue
Dcit: D-citrulline residue
DLys(AzaglyNic): D-(azaglysylnicotinoyl)lysine residue
DLys(AzaglyFur): D-(azaglysylfuranyl)lysine residue
DhArg(Et₂): D-(N,N′-diethyl)homoarginine residue
DAph(Atz): D-N-[5′-(3′-amino-1′H-1′,2′,4′-triazolyl)]phenylalanine residue
DhCi: D-homocitrulline residue
Lys(Nisp): (ε-N-isopropyl)lysine residue
hArg(Et₂): (N,N′-diethyl)homoarginine residue
D2Nal: D-3-(2-naphthyl)alanine residue

### DSer(tBu): O-tert-butyl-D-serine

Dhis(ImBzl): N^{im}-benzyl-D-histidine

When the other amino acids are represented by abbreviations, these are based on the abbreviations by IUPAC-IUB Commission of Biochemical Nomenclature (European Journal of Biochemistry, Vol. 138. pp. 9-37, 1984) or conventional abbreviations in the art. With regard to amino acids, when an optical isomer is exist, it represents L form unless otherwise mentioned.

### EXAMPLES

The present invention is illustrated in more detail with referring to the following Reference Example and Example, but these do not limit the present invention.

### Reference Example 1

An acetate of NAcD2NaI-D4ClPhe-D3 Pal-Ser-NMeTyr-DLys(Nic)-Leu-Lys(Nisp)-Pro-DAlaNH₂ (manufactured by TAP Pharmaceutical Products Inc., an LHRH antagonist compound) (480 mg) was added to a solution of a mixture of equal weight of glycolic acid (GA)-2-hydroxybutyric acid (HBA) copolymer (GA/HBA:60/40 (mol %), Mw: 16,300) and polylactic acid (Mw: 4,200) (3.52 g) in dichloromethane (4.5 mL) and dissolved. The solution was cooled to 16° C. and added to an aqueous solution of 0.1% polyvinyl alcohol (1,000 mL) which was previously adjusted to 16° C., and prepared an oil/water emulsion using a turbine-type homomixer at 7,000 rpm. The emulsion was stirred at room temperature for 3 hours to evaporate dichloromethane, and the oil phase was solidified and collected by a centrifuge (05PR-22, manufactured by Hitachi, Ltd.) at 2,000 rpm. This was dispersed again in distilled water and centrifuged to wash out the free drug and the like. Collected microcapsules were dispersed again by adding small amount of distilled water, then D-mannitol (1 g) was added to the dispersion and the dispersion was lyophilized to give powder. The percentage of the LHRH antagonist compound encapsulated in the microcapsules was 88%.

About 15 mg of the microcapsules were dispersed in a dispersion medium (distilled water in which 2.5 mg of carboxymethylcellulose, 0.5 mg of Polysolbate 80 and 25 mg of mannitol are dissolved) (0.5 mL) and the dispersion was administered subcutaneously to the dorsum of a 10 weeks-old male SD rat with a 22G injection needle (an administration dose as a LHRH antagonist: 3 mg/kg). After administration, blood was collected from the aorta inferior in each predetermined time and the rat was sacrificed, and the microcapsules remained at the site of administration were collected and the LHRH antagonist therein was quantified by HPLC, thereby confirmed that about 80% of the initial content of the LHRH antagonist compound was successively released for 4 weeks from the microcapsules. The concentration of testosterone in blood collected at each time was suppressed to not more than detection limit (0.05 ng/mL) for 4 weeks and recovered to about 50% of the mean value of the untreated normal rat (2.5 ng/mL) after 6 weeks, thereby the medicable period of the sustained release agent of the LHRH antagonist compound at this administration dose was determined to be 4 weeks.

### Example 1

Using 10 weeks-old male SD rat, the experiment was carried out for the following 4 groups. The second administration was carried out after 4 weeks of the first administration, and the testosterone in the serum of the collected blood before the second administration and after 1 day, 1 week, 2 weeks, 3 weeks, 4 weeks, 6 weeks, 8 weeks of the second administration was measured by RIA method.

| | Preparation of the | Preparation of the |
|---|---|---|
| | first administration | second preparation |
| Group I | No administration | Reference Example |
| Group | Leuplin injection | Reference Example |
| II | 3.75 | |
| Group | No administration | Leuplin injection |
| III | | 3.75 |
| Group | Reference Example | Leuplin injection |
| IV | | 3.75 |

The sustained release microcapsules of a LHRH antagonist for leuplin injection 3.75 (a one-month type sustained release microcapsules of an LHRH agonist, manufactured by Takeda Chemical Industries, Ltd., S130) obtained in Reference Example 1 was administrated subcutaneously by 3 mg/kg in terms of the respective compounds. The testosterone concentration below the detection limit of 0.05 ng/mL in the RIA, was regarded as 0.00 ng/mL.

Table 1 shows the average concentration of testosterone and standard deviation (SD, n=5) (unit: ng/mL).

TABLE 1

| | Group I | Group II | Group III | Group IV |
|---|---|---|---|---|
| before the second | 2.48 | 0.35 | 2.49 | 0.00 |
| administration | (0.38) | (0.12) | (0.41) | (0.00) |
| after one day of | 0.00 | 0.00 | 4.50 | 0.07 |
| the second | (0.00) | (0.00) | (0.60) | (0.03) |
| administration | | | | |
| after one week of | 0.00 | 0.00 | 0.30 | 0.00 |
| the second | (0.00) | (0.00) | (0.05) | (0.00) |
| administration | | | | |
| after two weeks of | 0.00 | 0.00 | 0.28 | 0.00 |
| the second | (0.00) | (0.00) | (0.05) | (0.00) |
| administration | | | | |
| after three weeks | 0.00 | 0.00 | 0.29 | 0.13 |
| of the second | (0.00) | (0.00) | (0.08) | (0.07) |
| administration | | | | |
| after four weeks | 0.00 | 0.00 | 0.35 | 0.22 |
| of the second | (0.00) | (0.00) | (0.12) | (0.08) |
| administration | | | | |
| after six weeks of | 1.26 | 1.02 | 0.52 | 0.53 |
| the second | (0.28) | (0.41) | (0.11) | (0.13) |
| administration | | | | |
| after eight weeks | 2.40 | 2.06 | 1.34 | 1.21 |
| of the second | (0.43) | (0.42) | (0.27) | (0.14) |
| administration | | | | |

The testosterone after 6 weeks or 8 weeks of the administration in the Groups I and II in which the sustained release microcapsules of a LHRH antagonist were administrated at the second administration, was more closer to the normal value of 2.5 ng/mL than that of the Groups III and IV in which the sustained release microcapsules of a LHRH agonist were administrated, and was more quickly recovered by administrating a preparation of the LHRH antagonist compound finally and the initiation of the drug cessation was more definitely determined. Then, the former administration mode is more suitable for an intermittent treatment.

### INDUSTRIAL APPLICABILITY

By using a preparation of the present invention which contains a compound having a luteinizing hormone-releasing hormone antagonistic effect or a salt thereof or a preparation containing the compound (preferably a sustained release preparation), the concentration of a sex hormone (e.g., testosterone, LH, FSH, estrogen and the like) after the medicable period of the compound having a luteinizing hormone-releasing hormone agonistic effect or a salt thereof or a preparation containing the compound (preferably as compared to the groups of a single treatment with a sustained release preparation) is recovered more quickly, which makes it possible to definitely determine the drug cessation period in an intermittent treatment.

1. A medicinal preparation for treating sex hormone-dependent diseases comprising a combination of a compound having a luteinizing hormone-releasing hormone agonistic effect or a salt thereof with a compound having a luteinizing hormone-releasing hormone antagonistic effect or a salt thereof, for administering the compound having a luteinizing hormone-releasing hormone agonistic effect or a salt thereof followed by the compound having a luteinizing hormone-releasing hormone antagonistic effect or a salt thereof.

2. The preparation according to claim 1, wherein the compound having a luteinizing hormone-releasing hormone agonistic effect or a salt thereof is a peptide compound or a salt thereof.

3. The preparation according to claim 2, wherein the peptide compound is a natural hormone or an analog thereof.

4. The preparation according to claim 2, wherein the peptide compound is a peptide represented by the formula:
5-oxo-Pro-His-Trp-Ser-Tyr-Y-Leu-Arg-Pro-Z
wherein Y is a residue selected from DLeu, DAla, DTrp, DSer(tBu), D2NaI and DHis(ImBzl), Z is NH—C₂H₅ or Gly-NH₂.

5. The preparation according to claim 2, wherein the peptide compound or a salt thereof is a compound or a salt thereof selected from leuprorelin, gonadorelin, buserelin, triptorelin, goserelin, nafarelin, histrelin, deslorelin, meterelin and recirelin.

6. The preparation according to claim 1, wherein the compound having a luteinizing hormone-releasing hormone agonistic effect or a salt thereof is used as an injectable preparation.

7. The preparation according to claim 1, wherein the compound having a luteinizing hormone-releasing hormone agonistic effect or a salt thereof is used as a sustained release preparation.

8. The preparation according to claim 1, wherein the compound having a luteinizing hormone-releasing hormone agonistic effect or a salt thereof is used as a preparation for nasal administration.

9. The preparation according to claim 1, wherein the compound having a luteinizing hormone-releasing hormone antagonistic effect or a salt thereof is a peptide compound or a salt thereof.

10. The preparation according to claim 9, wherein the peptide compound having a luteinizing hormone-releasing hormone antagonistic effect or a salt thereof is a compound selected from NAcD2NaI-D4ClPhe-D3 Pal-Ser-NMeTyr-DLys(Nic)-Leu-Lys(Nisp)-Pro-DAlaNH₂, N(4H2-furoyl)Gly-D2NaI-D4ClPhe-D3 Pal-Ser-NMeTyr-DLys(Nic)-Leu-Lys(Nisp)-Pro-DAlaNH₂, cetrorelix, ganirelix, antarelix, decirelix, azaline, antide, ramorelix and abarelix, or a salt thereof.

11. The preparation according to claim 1, wherein the compound having a luteinizing hormone-releasing hormone antagonistic effect or a salt thereof is used as an injectable preparation.

12. The preparation according to claim 1, wherein the compound having a luteinizing hormone-releasing hormone antagonistic effect or a salt thereof is used as a sustained release preparation.

13. The preparation according to claim 1, wherein the compound having a luteinizing hormone-releasing hormone antagonistic effect or a salt thereof is used as a preparation for nasal administration.

14. The preparation according to claim 1, wherein the compound having a luteinizing hormone-releasing hormone antagonistic effect or a salt thereof is a nonpeptide compound or a salt thereof.

15. The preparation according to claim 14, wherein the nonpeptide compound or a salt thereof is a compound represented by the formula (I): 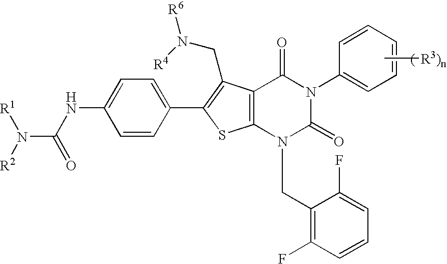
wherein each of R¹ and R² is hydrogen atom, hydroxy group, a C₁₋₄ alkoxy group, a C₁₋₄ alkoxy-carbonyl group or an optionally substituted C₁₋₄ alkyl group,
R³ is hydrogen atom, a halogen atom, hydroxy group or an optionally substituted C₁₋₄ alkoxy group, or the two adjacent R³s may link to form a C₁₋₄ alkylenedioxy group,
R⁴ is hydrogen atom or a C₁₋₄ alkyl group,
R⁶ is an optionally substituted C₁₋₄ alkyl group or a group represented by the formula: 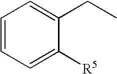
wherein R⁵ is hydrogen atom, or R⁴ and R⁵ may link to form a heterocyclic ring, and
n is an integer of 0 to 5, or a salt thereof.

16. The preparation according to claim 14, wherein the nonpeptide compound or a salt thereof is 5-(N-benzyl-N-methylaminomethyl)-1-(2,6-difluorobenzyl)-6-[4-(3-methoxyureido)phenyl]-3-phenylthieno[2,3-d]pyrimidine-2,4-(1H,3H)-dione or a salt thereof.

17. The preparation according to claim 14, wherein the nonpeptide compound or a salt thereof is a compound represented by the formula (VIII): 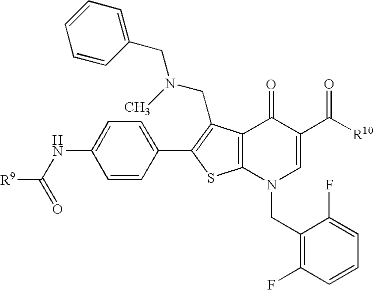
wherein R⁹ is an optionally substituted C₁₋₇ alkyl group, an optionally substituted C₃₋₇ cycloalkyl group, an optionally substituted C₁₋₆ alkoxyamino group or an optionally substituted hydroxyamino group,
R¹⁰ is an optionally substituted C₁₋₇ alkyl group or an optionally substituted phenyl group, or a salt thereof.

18. The preparation according to claim 14, wherein the nonpeptide compound or a salt thereof is 3-(N-benzyl-N-methylaminomethyl)-4,7-dihydro-5-isobutylyl-7-(2,6-difluorobenzyl)-2-[4-[(1-hydroxycyclopropyl)-carbonylamino]phenyl]-4-oxothieno[2,3-b]pyridine or a salt thereof.

19. The preparation according to claim 1, wherein the compound having a luteinizing hormone-releasing hormone antagonistic effect or a salt thereof is administrated before administration, during administration or immediately after administration of the compound having a luteinizing hormone-releasing hormone agonistic effect or a salt thereof.

20. A method for using a compound having a luteinizing hormone-releasing hormone agonistic effect or a salt thereof and/or a compound having a luteinizing hormone-releasing hormone antagonistic effect or a salt thereof for the preparation of a medicinal preparation for treating sex hormone-dependent diseases, said preparation being characterized by administering the compound having a luteinizing hormone-releasing hormone agonistic effect or a salt thereof, followed by the compound having a luteinizing hormone-releasing hormone antagonistic effect or a salt thereof.

21. A medicinal preparation for treating sex hormone-dependent diseases comprising a compound having a luteinizing hormone-releasing hormone agonistic effect or a salt thereof or a compound having a luteinizing hormone-releasing hormone antagonistic effect or a salt thereof, said preparation being characterized by administering the compound having a luteinizing hormone-releasing hormone agonistic effect or a salt thereof, followed by the compound having a luteinizing hormone-releasing hormone antagonistic effect or a salt thereof.

22. An agent for maintaining a hormone therapy comprising a compound having a luteinizing hormone-releasing hormone antagonistic effect or a salt thereof.

23. A method for determining a drug cessation period comprising using a compound having a luteinizing hormone-releasing hormone antagonistic effect or a salt thereof.

24. A method for recovering the concentration of a sex hormone in a living body comprising using a compound having a luteinizing hormone-releasing hormone antagonistic effect or a salt thereof.

25. A method for the treatment of sex hormone-dependent diseases comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal.

26. A method for maintaining hormone therapy comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal.

27. A method for determining a drug cessation period comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal.

28. A method for recovering sex hormone concentration in a living body comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal.

29. A method for treating a sex hormone-dependent disease, comprising administering an effective amount of a compound having a luteinizing hormone-releasing hormone agonistic effect or a salt thereof followed by an effective amount of a compound having a luteinizing hormone-releasing hormone antagonistic effect or a salt thereof to a mammal, thereby maintaining sex hormone-dependency.
